(19) United States
(12) Patent Application Publication
(10) Pub. No.: US 2005/0231687 A1
(43) Pub. Date: Oct. 20, 2005

(54) Edge detector in an ophthalmic eye evaluation system

(76) Inventor: Lloyd Allred, Bountiful, UT (US)

Correspondence Address: Bausch & Lomb Incorporated, One Bausch & Lomb Place, Rochester, NY, 14604-2701, US

(21) Appl. No.: 11/072,369

(22) Filed: Mar. 4, 2005

Related U.S. Application Data
(60) Provisional application No. 60/557,770, filed on Mar. 30, 2004.

(51) Int. Cl. A61B 3/10

(52) U.S. Cl. 351/212; 351/211; 351/221

(57) ABSTRACT

An ophthalmic eye evaluation system 10 includes a pattern 12 of alternating light and dark areas. An illumination source 14 projects the pattern 12 onto a patient's eye 16. A camera 18 captures one or more images of the pattern 12 reflected from the eye 16. A memory 20 is connected to the camera 18 for storing the images of the reflected pattern. An edge detector 22 determines a transition point where the stored reflected pattern alternates from a light area to a dark area. The edge is determined as being at a point 50% between a maximum stored intensity level and a minimum stored intensity level.

Priority is hereby claimed in the present nonprovisional application to Provisional Application Serial No. 60/557,770 filed Mar. 30, 2004, in accordance with 37 CFR 1.78(a)(4).

### BACKGROUND OF THE INVENTION

1. Field of the Invention

The present invention is related to ophthalmic eye evaluation systems, such as placido devices. More particular, the present invention is related to edge detection in such placido inventions for precisely locating a transition point between a light area and a dark area.

2. Description of Related Art

Placido devices are well known in the ophthalmic art for obtaining curvature data for a patient's eye, and in particularly the cornea. The standard prior art placido device includes illuminating a pattern of concentric rings onto a patient's eye. These rings are typically alternating white and black rings.

The point of transition from a light area to a dark area on a placido pattern theoretically, should be a step function that transitions directly from a white or light area to a black or dark area with no transition in between.

Historically these placidos images have been analyzed via the reflection off the patient's cornea and any deformation of the placido pattern from that projected onto the eye indicates an aberration in the curvature of the patient's eye. These aberrations can be observed manually by a physician or these placido patterns may be evaluated automatically by a diagnostic instrument. Such diagnostic instruments are well known in the art, and are offered by many companies for evaluation of a patient's eyes.

When these placido patterns are analyzed by a machine, they are typically captured by a video camera. The video camera captures a reflected image from the patient's cornea, and then a central processing unit with appropriate software converts the pixel data that is received from the video camera into charts and graphs that are useful to a physician. One of the essential elements in developing such charts and graphs, is determining the edges between the light and dark areas of the placido pattern, i.e., the precise point at which the pattern alternates from a light area to a dark area.

As stated above, under ideal conditions the transition should occur at all times at the same radius and yield a step function. This however, does not occur in practice because of a number of factors, including errors in fabrication, pixilation, and smearing from numerous optical effects, and various other noise factors. Other additional errors that may be introduced into the system may include lighting variations, pigment variations, reflections, and the sensor array. The sensor array introduces error because each pixel does not respond the same, and therefore, sensor noise is introduced into the system. Finally, in addition there is blooming, which is a condition where energy leaks between adjacent pixels.

Prior art techniques have used a maximum slope evaluation of light intensity from a transition from light to dark to determine an edge. Calculating the slope involves taking differences between different points on the image and such edge points can be somewhat noisy. These maximum slopes are typically taken from graphs formed from Hough transforms. Using the maximum slope prior art technique can often lead to identification errors because of the noise and spurious peak values can cause misidentification of spurious cross-over points.

Therefore, it would be desirable to have an edge detection technique that provides for more precise and accurate edge detection when determining the transition from a light area to a dark area or vice versa.

### BRIEF DESCRIPTION OF THE DRAWINGS

FIG. 1 is a partial block diagram of an ophthalmic system in accordance with the present invention;

FIG. 2 is a graph of Hough transform data obtained from a typical transition from a dark area to a light area;

FIG. 3 is a histogram of resulting edge values using the prior art maximum slope technique; and

FIG. 4 is a histogram of resulting edge values using a system in accordance with the present invention.

### DETAILED DESCRIPTION

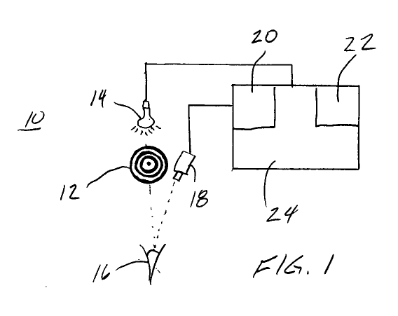

An ophthalmic eye evaluation system 10, in accordance with the present invention, is shown in FIG. 1. System 10 includes a pattern of alternating light and dark areas 12 and an illumination source 14 associated with the pattern 12 for projecting the pattern 12 onto a patient's eye 16. At least one camera 18 is positioned relative to the eye 16 for capturing one or more images of the pattern 12 reflected from the eye 16. A memory 20 is connected to the camera 18 for storing images of the reflected pattern. An edge detector 22 determines the transition point where the stored reflected pattern alternates from a light area to a dark area. The edge is determined as being at a point 50% between a maximum stored intensity level and a minimum stored intensity level.

Preferably memory 20 and edge detector 22 form a portion of a central processing unit 24.

While the pattern 12 is shown as a standard placido pattern of concentric rings, pattern 12 may also form other patterns, such as the spider-web pattern described in detail in U.S. patent application Ser. No. 10/261,539 filed 30 Sep. 2002, and entitled Spider-Web Placido Pattern, which application is incorporated herein by reference. In addition, other patterns are known, such as checkerboard patterns or the like. All the patterns have a common step transition from light to dark or dark to light areas.

By using the inventive 50% of rise technique described, more precise placido analyses were obtained as compared to the prior art maximum slope technique. Using the maximum slope technique, the results often were not even on the correct ring, making the results unusable. In order to compare the results between the two techniques, a graph of Hough transforms was obtained from a simulation, as shown in FIG. 2. A transition from a dark area shown generally at 26 to a light area shown generally at 28 is shown in FIG. 2. The simulation was performed using various levels of sensor noise, lighting variations, pixilation, optical blurring, and artifact clutter. The resulting edge curves of FIG. 2 has similar character to real data.

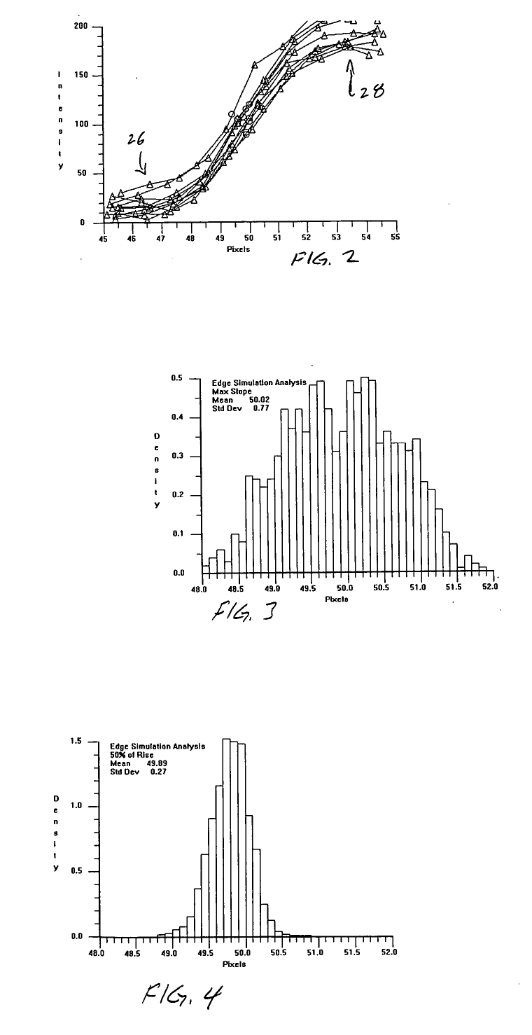

The original edge for all of the above curves was specified at 50 pixels. A simulation was performed for 1,000 curves, like those shown in FIG. 2. A histogram of the resulting edge values using the maximum slope technique is shown in FIG. 3. As can be seen from the graph, the mean value of the edge was determined to be at 50.02 pixels with a standard deviation or error of 0.77 pixels.

The histogram of FIG. 4 using the 50% of rise technique for edge analysis, shows a mean value of 49.89 pixels was achieved with a standard deviation of only 0.27 pixels. As can be seen from comparing the standard deviations—using the slope technique, a standard error of approximately three (3) times that of 50% of rise technique was experienced.

When using the 50% of rise technique, the accuracy is equivalent to the accuracy of the interpolated curve. Because the curve is interpolated, the amount of error is decreased. The slope method however, involves taking differences between different points on the image. These errors become additive, and the resulting edge points can be somewhat noisy. Finding the maximum slope involves fitting some kind of curve through the resulting slopes thus, resulting in even more error. In addition, because the present invention looks for 50% of rise, several points on the plateau and the floor can be used to get an accurate calibration of the light intensity, thus greatly reducing the measured noise. This is a sharp contrast to using slopes. Each slope calculation contains combined effects of the noise from two (2) measurements, thus doubling the measurement noise.

Thus, determining the edge and the transition from a light area to a dark area or visa versa, is simply a matter of taking an average floor value of a dark area which becomes a minimum intensity level and finding an average ceiling or maximum intensity level of a light area and determining a transition point or edge to be half way between or 50% of the difference between the maximum intensity and minimum intensity levels.

Typically in practice, the present invention would include one digital camera positioned relative to the eye for capturing one or more images of the pattern 12 reflected from the eye 16. The captured image is typically captured as an array of pixels where each pixel captures an illumination intensity level where a maximum intensity level corresponds to the pattern's 12 light areas and a minimum intensity level corresponds to the pattern's dark areas. The edge detector 22 then determines a transition point where the stored captured image alternates from a light area to a dark area wherein the edge is determined as being at a point 50% between the maximum intensity and minimum intensity levels of closely spaced pixels. Typically, these pixels are at least preferably two (2) pixels apart and preferably no more than 15 pixels apart.

Thus, has been shown an inventive ophthalmic eye evaluation system that more precisely determines an edge between a step function transition in a placido pattern from a light area to a dark area. Other variations to the present invention will be obvious to those skilled in the art and should be considered within the scope of the present invention such as the use of different types of cameras, memories, and illumination patterns, as well as different methods of illuminating a pattern onto a patient's eye.

1. An ophthalmic eye evaluation system comprising:
a pattern of alternating light and dark areas;
an illumination source associated with the pattern for projecting the pattern onto a patient's eye;
at least one camera positioned relative to the eye for capturing one or more images of the pattern reflected from the eye;
memory connected to the camera for storing the images of the reflected pattern; and
an edge detector for determining a transition point where the stored reflected pattern alternates from a light area to a dark area, wherein the edge is determined as being at a point 50% between a maximum stored intensity level and a minimum stored intensity level.

2. The invention of claim 1, wherein the pattern is a placido pattern.

3. The invention of claim 1, wherein the pattern is a spider-web like pattern.

4. An ophthalmic eye evaluation system comprising:
a pattern of alternating light and dark areas;
an illumination source associated with the pattern for projecting the pattern onto a patient's eye;
at least one digital camera positioned relative to the eye for capturing one or more images of the pattern reflected from the eye wherein the captured image is captured as an array of pixels where each pixel captures an illumination intensity level where a maximum intensity level corresponds to the pattern's light areas and a minimum intensity level corresponds to the pattern's dark area;
a memory connected to the camera for storing the captured images; and
an edge detector for determining a transition point where the stored captured image alternates from a light area to a dark area, wherein the edge is determined as being at a point 50% between the maximum intensity and minimum intensity of closely spaced pixels.

5. The invention of claim 4, wherein the pattern is a placido pattern.

6. The invention of claim 4, wherein the pattern is a spider-web like pattern.

7. The invention of claim 4, wherein the distance between the maximum and minimum intensity pixels is at least 2 pixels.

8. The invention of claim 4, wherein the distance between the maximum and minimum intensity pixels is no more than 15 pixels.
